(19) United States
(12) Patent Application Publication
(10) Pub. No.: US 2006/0165671 A1
(43) Pub. Date: Jul. 27, 2006

(54) Novel nutraceutical compositions comprising epigallocatechin gallate

(76) Inventors: Daniel Raederstorff, Brunstatt (FR); Sandra Renata Teixeira, Brookline, MA (US); Peter Weber, Malsburg-Marzell (DE)

Correspondence Address: Bryan Cave, 1290 Avenue of the Americas, New York, NY, 10104, US

(21) Appl. No.: 10/533,858

(22) PCT Filed: Sep. 30, 2003
(86) PCT No.: PCT/EP03/10838

§ 371 Date: Dec. 12, 2005

(30) Foreign Application Priority Data
Nov. 7, 2002 (EP) 02024804.3

(51) Int. Cl. A61K 38/43; A61K 31/385; A61K 31/20; A61K 31/045; A61K 36/82

(52) U.S. Cl. 424/94.1; 424/729; 514/440; 514/558; 514/724

(57) ABSTRACT

The invention relates to nutraceutical compositions comprising at least two ingredients from the groups of EGCG, pantethine or a metabolite thereof, phytanic acid, lipoic acid, policosanol and coenzyme Q-10 and their use in the treatment or prevention of diabetes or obesity.

The present invention relates to novel nutraceutical compositions comprising at least two components selected from EGCG, pantethine or a metabolite thereof, phytanic acid, lipoic acid, policosanol and coenzyme Q-10 as the active ingredients for the treatment or prevention of diabetes mellitus, or other conditions associated with impaired glucose tolerance such as syndrome X and obesity. In another aspect the present invention relates to the use of such compositions as a nutritional supplement for the said treatment or prevention, e.g., as an additive to a multi-vitamin preparations comprising vitamins and minerals which are essential for the maintenance of normal metabolic function but are not synthesized in the body. In still another aspect, the invention relates to a method for the treatment of both type 1 and 2 diabetes, and for the prevention of type 2 diabetes in those individuals with pre-diabetes, or impaired glucose tolerance (IGT) or obesity which comprises administering to a subject in need of such treatment at least two components selected from EGCG, pantethine or a metabolite thereof, phytanic acid, lipoic acid, policosanol and coenzyme Q-10.

The compositions of the present invention are particularly intended for the treatment of both type 1 and 2 diabetes, and for the prevention of type 2 diabetes in those individuals with pre-diabetes, or impaired glucose tolerance (IGT), or obesity.

The compositions comprising a combination of active ingredients, i.e., at least two components selected from EGCG, pantethine or a metabolite thereof, phytanic add, lipoic acid, policosanol and coenzyme Q-10 have different mechanism of action on glucose metabolism and insulin sensitivity thus providing additive and/or synergetic effects in the treatment of diabetes.

The term nutraceutical as used herein denotes a usefulness in both the nutritional and pharmaceutical field of application. Thus, the novel nutraceutical compositions can find use as supplement to food and beverages, and as pharmaceutical formulations for enteral or parenteral application which maybe solid formulations such as capsules or tablets, or liquid formulations, such as solutions or suspensions. As will be evident from the foregoing, the term nutraceutical composition also comprises food and beverages containing at least two components selected from EGCG, pantethine or a metabolite thereof, phytanic acid, lipoic acid, policosanol and coenzyme Q-10, as well as supplement compositions containing the aforesaid active ingredients.

Diabetes is a widespread chronic disease that hitherto has no cure. The incidence and prevalence of diabetes is increasing exponentially and it is among the most common metabolic disorder in developed and developing countries. Diabetes mellitus is a complex disease derived from multiple causative factors and characterized by impaired carbohydrate, protein and fat metabolism associated with a deficiency in insulin secretion and or insulin resistance. This results in elevated fasting and postprandial serum glucose that leads to complications if left untreated. There are two major categories of the diseases, insulin-dependent diabetes mellitus (IDDM, type 1) and non-insulin-dependent diabetes mellitus (NIDDM, type 2).

Type 1 and type 2 diabetes are associated with hyperglycemia, hypercholesterolemia and hyperlipidemia. The insensitivity to insulin and absolute insulin deficiency in type 1 and 2 diabetes leads to a decrease in glucose utilization by the liver, muscle and the adipose tissue and to an increase in the blood glucose levels. Uncontrolled hyperglycemia is associated with increased and premature mortality due to an increased risk for microvascular and macrovascular diseases, including nephropathy, neuropathy, retinopathy, hypertension, stroke, and heart disease. Recent evidence showed that tight glycemic control is a major factor in the prevention of these complications in both type 1 and type 2 diabetes mellitus. Therefore, optimal glycemic control by drugs or therapeutic regimens is an important approach for the treatment of diabetes.

Therapy of type 2 diabetes initially involves dietary and lifestyle changes, when these measures fail to maintain adequate glycemic control the patients are treated with oral hypoglycemic agents and/or exogenous insulin. The current oral pharmacological agents for the treatment of type 2 diabetes mellitus include those that potentiate insulin secretion (sulphonylurea agents), those that improve the action of insulin in the liver (biguanide agents), insulin sensitizing agents (thiazolidinediones) and agents which act to inhibit the uptake of glucose (α-glucosidase inhibitors). However, currently available agents generally fail to maintain adequate glycemic control in the long term due to progressive deterioration in hyperglycaemia, resulting from progressive loss of pancreatic cell function. The proportion of patients able to maintain target glycemic levels decreases markedly overtime necessitating the administration of additional/alternative pharmacological agents. Furthermore, the drugs may have unwanted side effects and are associated with high primary and secondary failure rates. Finally, the use of hypoglycemic drugs may be effective in controlling blood glucose levels, but may not prevent all the complications of diabetes. Thus, current methods of treatment for all types of diabetes mellitus fail to achieve the ideals of normoglycemia and the prevention of diabetic complications. Therefore, although the therapies of choice in the treatment of type 1 and type 2 diabetes are based essentially on the administration of insulin and of oral hypoglycemic drugs, there is a need for a safe and effective nutritional supplement with minimal side effects for the treatment and prevention of diabetes. Many patients are interested in alternative therapies which could minimize the side effects associated with high-dose of drugs and yield additive clinical benefits. Patients with diabetes have a special interest in treatment considered as “natural” with mild anti-diabetic effects and without major side effects, which can be used as adjuvant treatment. Type 2 diabetes is a progressive and chronic disease, which usually is not recognized until significant damage has occurred to the pancreatic cells responsible for producing insulin. Therefore, there is also an increasing interest in the development of a dietary supplement that may be used to prevent the development of diabetes in people at risk especially in elderly who are at high risk for developing diabetes. Furthermore, type 2 is a complicated disease resulting from coexisting defects at multiple organ sites: resistance to insulin action in muscle and adipose tissues, defective pancreatic insulin secretion, unrestrained hepatic glucose production associated with lipid abnormalities and endothelial dysfunction. Therefore, given the multiple pathophysiological lesions in type 2 diabetes, combination therapy is an attractive approach to its management.

The use of combinations of EGCG, pantethine or a metabolite thereof, Coenzyme Q-10, phytanic acid, policosanol and/or lipoic acid which individually exert different mechanisms of action are effective in achieving and maintaining target blood glucose levels in diabetic patients.

The combinations of the active ingredients identified above have been conceived because of their different actions, to take advantage of additive/synergetic and multiorgan effects. Owing to distinct mechanism of action of the individual active ingredients the combinations not only improve glycemic control, but also result in lower drug dosing in some settings and minimize adverse effects. Because of their distinct mechanism and sites of action, the specific combinations of dietary supplements discussed above also take advantage of additive/synergetic effects to achieve a degree of glucose lowering greater than single agents can accomplish. Thus, although the therapies of choice in the therapeutic treatment of type 1 and type 2 diabetes is based essentially on the administration of insulin and of oral hypoglycemic drugs appropriate nutritional therapy is also of major importance for the successful treatment of diabetics.

The function of each of the active ingredients of the nutraceutical compositions of the present invention is described below.

EGCG:

Epigallocatechin gallate (EGCG) is the major catechin found in green tea. In rats green tea catechins dose-dependently suppressed the increase in glucose and insulin levels in plasma after a starch or a sucrose rich meal. Combinations of EGCG and pantethine or phytanic acid according to the invention are especially useful for patients who have impaired glucose tolerance, older patients who develop an increase in postprandial glucose due to aging, and patients with undiagnosed diabetes.

Pantethine:

In human studies oral administration of pantethine resulted in a progressive decrease in total cholesterol, triglycerides, low density lipoprotein (LDL) cholesterol and an increase in high density lipoprotein (HDL) cholesterol. Thus, resulting in a more favorable Chol/HDL ratio which reduces cardiovascular risk. Diabetes mellitus is associated with a 3- to 4-fold increase in risk of coronary artery disease. Type 2 diabetes mellitus adversely affects the plasma lipid profile, increasing levels of atherogenic lipids such as low density lipoproteins (LDL) and very low density lipoproteins (VLDL), but decreasing levels of high density lipoprotein (HDL), an antiatherogenic lipid. Atherosclerotic manifestations are not only common in individuals with diabetes but also result in significant long-term complications. Therefore, the oral supplementation with pantethine helps diabetes patients to normalize their lipid values reducing the risk of coronary heart disease and of thrombotic events. Instead of or in addition to panthethine, metabolites of pantethine such as cysteamine and pantothenic acid may find use in accordance with the invention.

Lipoic Acid:

Lipoic acid (1,2-dithiolane-3-pentaenoic acid) plays an essential role in mitochondrial-specific pathways that generate energy from glucose and may potentially influence the rate of glucose oxidation. Lipoic acid stimulates glucose transport in both muscle and adipose cells in culture. Moreover, administration of lipoic add also raised basal and insulin-stimulated glucose uptake by skeletal muscles of glucose intolerant and non-insulin dependent diabetic animals. Furthermore, lipoic acid improves glucose disposal in patients with type 2 and maybe incorporated in a nutraceutical composition of the present invention in order to prevent and/or treat the diabetic related complications and as agent with insulin sensitizing activity.

Phytanic Add:

Phytanic acid (3,7,11,15-tetramethylhexadecanoic acid) at concentrations ranging from about 10 to about 100 μM enhances uptake of glucose in rat primary hepatocytes. Compared to the specific PPAR-γ agonist such as ciglitazone, phytanic acid exerts only minor effects on the differentiation of pre-adipocyte cells into mature adipocytes. Therefore, intake of phytanic acid helps to improve insulin sensitivity and may act as a preventative measure against type 2 diabetes and Syndrome X through activation of PPARs and RXR.

Coenzyme Q-10:

Coenzyme Q-10, (6-Decaprenyl-2,3-dimethoxy-5-methyl-1,4-benzoquinone) is a fat soluble quinone with a structure similar to vitamin K. The health beneficial effects of Coenzyme Q10 (CoQ10) have been associated with its two main biochemical functions. CoQ10 is an essential cofactor of the mitochondrial electron transport chain which, is coupled to synthesis of adenosine triphosphate (ATP). Therefore, it acts as a catalyst in the biochemical pathway that leads to cellular energy production. This bioenergic effect of CoQ10 is of particular importance in cells with high metabolic demands such as cardiac myocytes. Moreover, CoQ10 is an important antioxidant in both the mitochondria and lipid membranes. CoQ10 exerts a sparing effect on vitamin E and has membrane stabilizing properties. Several studies showed that LDL oxidation was reduced after CoQ10 supplementation. Thus CoQ10 may improve energy metabolism and protect against oxidative stress in diabetes and cardiovascular diseases.

Policosanol:

Policosanol is a mixture of primary aliphatic alcohols isolated and purified from plant waxes, mainly sugar cane. The aliphatic alcohol of the mixture is a CH₃—(CH₂)ₙ—CH₂OH alcohol with chain length varying from 18 to 40 carbon atoms. Typical aliphatic alcohols of the mixture are octacosanol, hexacosanol, heptacosanol, triacontanol and dotriacontanol. Policosanol has been shown to lower cholesterol in animal models, healthy volunteers, and patients with type II hypercholesterolemia. Therefore, it is useful in the dyslipidemia associated with type 2 diabetes mellitus.

A multi-vitamin and mineral supplement maybe added to the nutraceutical compositions of the present invention to obtain an adequate amount of an essential nutrient missing in some diets. The multi-vitamin and mineral supplement may also be useful for disease prevention and protection against nutritional losses and deficiencies due to lifestyle patterns and common inadequate dietary patterns sometimes observed in diabetes. Moreover, oxidant stress has been implicated in the development of insulin resistance. Reactive oxygen species may impair insulin stimulated glucose uptake by disturbing the insulin receptor signaling cascade. The control of oxidant stress with antioxidants such as α-tocopherol (vitamin E) ascorbic acid (vitamin C) may be of value in the treatment of diabetes. Therefore, the intake of multi-vitamin supplement may be added to the above mentioned active substances to maintain a good balanced nutrition.

In a preferred aspect of the invention, the nutraceutical composition of the present invention contains EGCG which suitably is present in the composition according to the invention in an amount to provide a daily dosage from about 0.3 mg per kg body weight to about 30 mg per kg body weight of the subject to which it is to be administered. A food or beverage suitably contains about 5 mg per serving to about 500 mg per serving of EGCG. If the nutraceutical composition is a pharmaceutical formulation such formulation may contain EGCG in an amount from about 10 mg to about 500 mg per dosage unit, e.g., per capsule or tablet, or from about 20 mg per daily dose to about 2000 mg per daily dose of a liquid formulation.

In another preferred aspect of the invention, the nutraceutical composition of the present invention further contains pantethine. The amount of pantethine in the composition may be such to provide a daily dosage from about 1 mg per kg body weight to about 50 mg per kg body weight of the subject to which it is to be administered. A food or beverage suitably contains about 20 mg per serving to about 800 mg per serving of pantethine. If the nutraceutical composition is a pharmaceutical formulation such formulation may contain pantethine in an amount from about 20 mg to about 1000 mg per dosage unit, e.g., per capsule or tablet, or from about 70 mg per daily dose to about 3500 mg per daily dose of a liquid formulation.

If phytanic acid is present in the nutraceutical composition according to the invention its amount may be such to provide a daily dosage from about 1 mg per kg body weight to about 100 mg per kg body weight of the subject to which it is to be administered. A food or beverage suitably contains about 20 mg per serving to about 2000 mg per serving of phytanic acid. If the nutraceutical composition is a pharmaceutical formulation such formulation may contain phytanic acid in an amount from about 30 mg to about 500 mg per dosage unit, e.g., per capsule or tablet, or from about 70 mg per daily dose to about 7000 mg per daily dose of a liquid formulation. Phytanic add may also be used in the form of a biologically equivalent derivative thereof, such as an ester, e.g. the methyl or ethyl ester.

If lipoic acid is present in the nutraceutical composition according to the invention its amount may be such to provide a daily dosage from about 0.3 mg per kg body weight to about 30 mg per kg body weight of the subject to which it is to be administered. A food or beverage suitably contains about 5 mg per serving to about 500 mg per serving of lipoic acid. If the nutraceutical composition is a pharmaceutical formulation such formulation may contain lipoic acid in an amount from about 5 mg to about 800 mg per dosage unit, e.g., per capsule or tablet, or from about 5 mg per daily dose to about 2000 mg per daily dose of a liquid formulation.

If Coenzyme Q-10 is present in the nutraceutical composition according to the invention its amount may be such to provide a daily dosage from about 0.01 mg per kg body weight to about 30 mg per kg body weight of the subject to which it is to be administered. A food or beverage suitably contains about 1 mg per serving to about 400 mg per serving of CoQ10. If the nutraceutical composition is a pharmaceutical formulation such formulation may contain CoQ10 in an amount from about 1 mg to about 500 mg per dosage unit, e.g., per capsule or tablet, or from about 1 mg per daily dose to about 2000 mg per daily dose of a liquid formulation.

If policosanol is present in the nutraceutical composition according to the invention its amount may be such to provide a daily dosage from about 0.002 mg per kg body weight to about 1.5 mg per kg body weight of the subject to which it is to be administered. A food or beverage suitably contains about 0.1 mg per serving to about 20 mg per serving of policosanol. If the nutraceutical composition is a pharmaceutical formulation such formulation may contain policosanol in an amount from about 0.1 mg to about 30 mg per dosage unit, e.g.; per capsule or tablet, or from about 0.1 mg per daily dose to about 100 mg per daily dose of a liquid formulation.

Preferred nutraceutical compositions of the present invention comprise combinations of at least two components selected from EGCG, pantethine or a metabolite thereof, phytanic acid, lipoic acid and coenzyme Q-10, more particularly EGCG, panthetine, phytanic acid and Coenzyme Q-10, especially the combinations of
EGCG and pantethine;
EGCG and phytanic acid;
Pantethine and phytanic add;
EGCG and Coenzyme Q-10;
EGCG, phytanic add and Coenzyme Q-10;
EGCG, phytanic acid and pantethine; and
EGCG, phytanic acid, pantethine and Coenzyme Q-10.

Most preferred are the combinations of EGCG and pantethine or phytanic acid, and the combination of pantethine and phytanic acid.

Dosage Ranges (for a 70 kg Person)

EGCG: 20-2100 mg/day
Pantethine: 70-3500 mg/day
Phytanic acid: 70-7000 mg/day
Coenzyme Q-10: 1-2100 mg/day
Lipoic acid: 20-2100 mg/day
Policosanol: 0.15-100 mg/day

The following Examples illustrate the invention further.

A. Pharmaceutical compositions may be prepared by conventional formulation procedures using the ingredients specified below:

### EXAMPLE 1

### Soft Gelatin Capsule

Soft gelatin capsules are prepared by conventional procedures using ingredients specified below:
Active ingredients: EGCG 300 mg Pantethine 100 mg
Other ingredients: glycerol, water, gelatine, vegetable oil

### EXAMPLE 2

### Hard Gelatin Capsule

Hard gelatin capsules are prepared by conventional procedures using ingredients specified below.
Active ingredients: EGCG 150 mg Pantethine 100 mg
   Other Ingredients:
Fillers: lactose or cellulose or cellulose derivatives q.s
Lubricant: magnesium sterate if necessary (0.5%)

### EXAMPLE 3

### Tablet

Tablets are prepared by conventional procedures using ingredients specified below:
Active ingredients: EGCG 100 mg, pantethine 50 mg
Other ingredients: microcrystalline cellulose, silicone dioxide (siO2), magnesium stearate, crosscarmellose sodium.

B. Food items may be prepared by conventional procedures using ingredients specified below:

### EXAMPLE 4

### Soft Drink with 30% Juice

Typical serving: 240 ml
   Active Ingredients:

EGCG and one or more additional components selected from pantethine, Coenzyme Q-10, phytanic acid and lipoic acid are incorporated in this food item
Pantethine: 20-800 mg/per serving
EGCG: 5-500 mg/per serving
Phytanic acid: 20-2000 mg/per serving
Coenzyme Q-10: 1-400 mg/per serving
Lipoic acid: 5-500 mg/per serving
Policosanol: 0.1-20 mg/per serving

I. A Soft Drink Compound is Prepared from the Following Ingredients:

Juice concentrates and water soluble flavours

| | [g] |
|---|---|
| 1.1 Orange concentrate | |
| 60.3° Brix, 5.15% acidity | 657.99 |
| Lemon concentrate | |
| 43.5° Brix, 32.7% acidity | 95.96 |
| Orange flavour, water soluble | 13.43 |
| Apricot flavour, water soluble | 6.71 |
| Water | 26.46 |
| 1.2 Color | |
| β-Carotene 10% CWS | 0.89 |
| Water | 67.65 |
| 1.3 Acid and Antioxidant | |
| Ascorbic acid | 4.11 |
| Citric acid anhydrous | 0.69 |
| Water | 43.18 |
| 1.4 Stabilizers | |
| Pectin | 0.20 |
| Sodium benzoate | 2.74 |
| Water | 65.60 |
| 1.5 Oil soluble flavours | |
| Orange flavour, oil soluble | 0.34 |
| Orange oil distilled | 0.34 |

1.6 Active Ingredients

Active ingredients (this means the active ingredient mentioned above: EGCG and one or more of pantethine, Coenzyme Q-10, lipoic acid, policosanol and/or phytanic acid) in the concentrations mentioned above

Fruit juice concentrates and water soluble flavours are mixed without incorporation of air. The color is dissolved in deionized water. Ascorbic acid and citric acid is dissolved in water. Sodium benozoate is dissolved in water. The pectin is added unter stirring and dissolved while boiling. The solution is cooled down. Orange oil and oil soluble flavours are premixed. The active ingredients as mentioned under 1.6 are dry mixed and then stirred preferably into the fruit juice concentrate mixture (1.1).

In order to prepare the soft drink compound all parts 3.1.1 to 3.1.6 are mixed together before homogenising using a Turrax and then a high-pressure homogenizer (p₁=200 bar, P₂=50 bar).

II. A Bottling Syrup is Prepared from the Following Ingredients:

| | |
|---|---|
| Softdrink compound | 74.50 |
| Water | 50.00 |
| Sugar syrup 60° Brix | 150.00 |

The ingredients of the bottling syrup are mixed together. The bottling syrup is diluted with water to 1 l of ready to drink beverage.

Variations:

Instead of using sodium benzoate, the beverage may be pasteurised. The beverage may also be carbonised.

### EXAMPLE 5

### 5 Cereal Bread

Typical serving: 50 g
   Active Ingredients:

EGCG and one or more additional components selected from pantethine, Coenzyme Q-10, phytanic acid and lipoic acid are incorporated in this food items
Pantethine: 20-800 mg/per serving
EGCG: 5-500 mg/per serving
Phytanic acid: 20-2000 mg/per serving
Lipoic acid: 5-500 mg/per serving
Coenzyme Q-10: 1-400 mg/per serving

Policosanol: 0.1-20 mg/per serving

| Other components: | [%] |
|---|---|
| 5 cereal flour | 56.8 |
| Water | 39.8 |
| Yeast | 2.3 |
| Salt | 1.1 |

The yeast is dissolved in a part of the water. All ingredients are mixed together to form a dough. Salt is added at the end of the kneading time. After fermentation, the dough is reworked and divided before a loaf is formed. Before baking, the surface of the loaf is brushed with water and sprinkled with flour.

Procedure:

| | |
|---|---|
| Kneading: | |
| Spiral kneading system | 4 min 1^{st} gear, 5 min 2^{nd} gear |
| Dough proofing: | 60 min |
| Dough temperature: | 22-24° C. |
| Proofing time: | 30 min |
| Baking: | |
| Oven: | Dutch type oven |
| Baking temperature: | 250/220° C. |
| Baking time: | 50-60 min |

### EXAMPLE 6

### Cookies Type Milano

Typical serving: 30 g
   Active Ingredients:

EGCG and one or more additional components selected from pantethine, Coenzyme Q-10, phytanic acid and lipoic acid are incorporated in this food items
Pantethine: 20-800 mg/per serving
EGCG: 5-500 mg/per serving
Phytanic acid 20-2000 mg/per serving
Coenzyme Q-10: 1-400 mg/per serving
Lipoic acid: 5-500 mg/per serving

Policosanol: 0.1-20 mg/per serving

| Other components: | [g] |
|---|---|
| Wheat Flour, type 550 | 41.0 |
| Sugar | 20.5 |
| Fat/Butter | 20.5 |
| Whole egg (liquid) | 18.0 |
| Lemon Flavour | q.s. |
| Baking agent | q.s. |

All ingredients are added slowly under mixing to form a sweet short pastry.

Afterwards, the pastry is kept cool (4° C.) for at least 2 hours before flattening the pastry to a thickness of approx. 5 mm. Pieces are cut out and brushed with egg yolk on the surface before baking.

Baking:

| Oven: | fan oven | |
|---|---|---|
| Baking temperature: | 180° | C. |
| Baking time: | 15 | min |

### EXAMPLE 7

### Toast

Typical serving: 100 g
   Active Ingredients:

EGCG and one or more additional components selected from pantethine, Coenzyme Q-10, phytanic acid and lipoic acid are incorporated in this food items
Pantethine: 20-800 mg/per serving
EGCG: 5-500 mg/per serving
Phytanic acid: 20-2000 mg/per serving
Coenzyme Q-10: 1-400 mg/per serving
Lipoic acid: 5-500 mg/per serving

Policosanol: 0.1-20 mg/per serving

| Other components: | [%] |
|---|---|
| Wheat Flour, type 550 | 55.4 |
| Water | 33.2 |
| Yeast | 2.8 |
| Salt | 1.1 |
| Fat/Butter | 5.5 |
| Malt | 0.6 |
| Emulsifier baking agent | 1.4 |

The yeast is dissolved in a part of the water. All ingredients are mixed together to form a dough. Salt is added at the end of the kneading time. Afterwards, the dough is reworked, divided and placed in a baking tin for fermentation. After baking, the loaf is unmoulded directly.

Procedure:

| | |
|---|---|
| Kneading: | |
| Spiral kneading system | 5-6 min 1^{st} gear; 3-4 min 2^{nd} gear |
| Dough proofing: | none |
| Dough temperature: | 22-24° C. |
| Proofing time: | 40 min |
| Baking: | |
| Oven: | Dutch type oven |
| Baking temperature: | 220° C. |
| Baking time: | 35-40 min |

### EXAMPLE 8

### Yoghurt—Set Type; 3.5% Fat

Typical serving: 225 g
   Active Ingredients:

EGCG and one or more additional components selected from pantethine, EGCG, phytanic acid and lipoic add are incorporated in this food items
Coenzyme Q-10: 1-400 mg/per serving
Pantethine: 20-800 mg/per serving
EGCG: 5-500 mg/per serving
Phytanic acid: 20-2000 mg/per serving
Lipoic acid: 5-500 mg/per serving

Policosanol: 0.1-20 mg/per serving

| Other components: | [%] |
|---|---|
| Full fat milk (3.8% fat) | 90.5 |
| Skimmed milk powder | 2.0 |
| Sugar | 5.0 |
| Culture | 2.5 |

The milk is heated to 35° C. before addition of milk powder, stabiliser, sugar and active ingredients. This mixture is heated to 65° C. to dissolve all ingredients. Then the mixture is homogenized in a high-pressure homogenizer (p₁=150 bar, p₂=50 bar) at 65° C. This emulsion is then pasteurised at 80° C. for 20 minutes. After cooling to 45° C. natural yoghurt/culture is added and mixed. Then this mixture is filled into cups and fermented at 45° C. for 3-4 hours until a pH of 4.3 is reached and then stored at 4° C.

### EXAMPLE 9

### Yoghurt—Stirred Type; 3.5% Fat

Typical serving: 225 g

EGCG and one or more additional components selected from pantethine, Coenzyme Q-10, phytanic acid and lipoic acid are incorporated in this food items:
Coenzyme Q-10: 1-400 mg/per serving
Pantethine: 20-800 mg/per serving
EGCG: 5-500 mg/per serving
Phytanic acid: 20-2000 mg/per serving
Lipoic acid: 5-500 mg/per serving

Policosanol: 0.1-20 mg/per serving

| Other components: | [%] |
|---|---|
| Full fat milk (3.8% fat) | 90.2 |
| Skimmed milk powder | 2.0 |
| Stabiliser | 0.3 |
| Sugar | 5.0 |
| Culture | 2.5 |

The milk is heated to 35° C. before addition of milk powder, stabiliser, sugar and active ingredients. This mixture is heated to 65° C. to dissolve all ingredients before homogenisation in a high-pressure homogenizer (p₁=150 bar, p₂=50 bar) at 65° C. This emulsion is then pasteurised at 80° C. for 20 minutes. After cooling to 45° C. natural yoghurt/culture is added and mixed, followed by fermentation at 45° C. for 3-4 hours until a pH of 4.3 is reached. After cooling and stirring vigorously, the yoghurt is filled in cups and stored at 4° C.

### EXAMPLE 10

### Ice Cream; 8% Fat

Typical serving: 85 g
   Active Ingredients:

EGCG and one or more additional components selected from pantethine, Coenzyme Q-10, phytanic acid and lipoic acid are incorporated in this food items
Coenzyme Q-10: 1-400 mg/per serving
Pantethine: 20-800 mg/per serving
EGCG: 5-500 mg/per serving
Phytanic acid: 20-2000 mg/per serving
Lipoic acid: 5-500 mg/per serving

Policosanol: 0.1-20 mg/per serving

| Other components: | [g] |
|---|---|
| Milk (3.7% fat) | 600.00 |
| Cream (35% fat) | 166.00 |
| Skim milk powder | 49.10 |
| Sugar | 109.00 |
| Glucose syrup 80% | 70.00 |
| Ice cream stabiliser | 5.00 |
| Flavor | q.s. |
| Color | q.s |

Sugar, skim milk powder and stabiliser are added to the milk and cream, mixed and heated to 45° C. Then the colour as stock solution and the glucose syrup is added as well as the active ingredients. The mix is heated up and pasteurized (20 min, 80° C.). Then a homogenization step takes place. Afterwards the mix is cooled down under constant stirring and the flavour is added at 5° C. The mix maturated at 5° C. during at least 4 h and then passed through an the ice cream machine (overrun ca. 100%). The ice cream is filled into cups and stored at −20 to −30° C.

### EXAMPLE 11

### Wine Gums

Active Ingredients:

EGCG and one or more additional components selected from pantethine, EGCG, phytanic acid and lipoic acid are incorporated in this food items
Coenzyme Q-10: 1-400 mg/per 30 g
Pantethine: 20-800 mg/per 30 g
EGCG: 5-500 mg/per 30 g
Phytanic acid: 20-2000 mg/per 30 g
Lipoic acid: 5-500 mg/per 30 g

Policosanol: 0.1-20 mg/per serving

| Other components: | [g] |
|---|---|
| Gelatine 200 Bloom | 80.0 |
| Water I | 125.0 |
| Sugar crys. | 290.0 |
| Water II | 120.0 |
| Glucose-syrup DE 38 | 390.0 |
| Citric acid | 10.0 |
| Flavour | 2.0 |
| Colour | q.s. |
| Yield ca | 1000.0 |

Disperse gelatine in water I, stir and dissolve by heating over a stream bath or using a microwave. Mix sugar with water II and bring to boiling until a clear solution is obtained. Remove from heat source. Mix with glucose syrup while dissolved sugar solution is still hot. Slowly add the gelatine solution. Let rest until foam on surface can be removed and 60-65° C. is reached. Add flavour, citric acid and the colour solution as well as active ingredients under stirring. Deposit into moulds printed into starch trays and let sit for at least 48 hours at RT. Remove starch powder and polish with oil or wax. Dry at RT and package into airtight pouches

1. A composition for the treatment or prevention of type 2 diabetes in those individuals with pre-diabetes, or impaired glucose tolerance (IGT) or obesity comprising EGCG and at least one of pantethine or phytanic acid.

2. A composition according to claim 1 comprising EGCG and pantethine.

3. A composition according to claim 1 comprising EGCG and phytanic acid.

4. A composition according to claim 1 comprising EGCG, pantethine and phytanic acid.

5. A composition according to claim 1 containing EGCG in an amount sufficient to administer to a subject a daily dosage of 0.3 mg per kg body weight to about 30 mg per kg body weight.

6. A composition according to claim 1 containing pantethine in an amount sufficient to administer to a subject a daily dosage of 1 mg per kg body weight to about 50 mg per kg body weight.

7. A composition according to claim 1 containing phytanic acid in an amount sufficient to administer to a subject a daily dosage of 1 mg per kg body weight to about 100 mg per kg body weight.

8. A composition according to claim 1 which is in dosage unit form.

9. A composition as in claim 8 wherein the dosage unit form is a solid dosage unit form.

10. A composition as in claim 9 wherein the dosage unit form contains about 10 mg to about 500 mg of EGCG.

11. A composition as in claim 9 wherein the dosage unit form contains about 20 mg to about 1000 mg of pantethine.

12. A composition as in claim 9 wherein the dosage unit form contains about 30 mg to about 500 mg of phytanic acid.

13. A composition according to claim 1 which is a food or beverage or a supplement composition for a food or beverage.

14. A food or beverage comprising EGCG, pantethine or a metabolite thereof, phytanic acid, lipoic acid, policosanol and coenzyme Q-10.

15-19. (canceled)

20. A method for the treatment of both type 1 and 2 diabetes, and for the prevention of type 2 diabetes in those individuals with pre-diabetes, or impaired glucose tolerance (IGT) or obesity which comprises administering to a subject in need of such treatment EGCG and at least one of pantethine and phytanic acid.

21. A method of making a nutraceutical composition comprising admixing EGCG and at least one of pantethine or phytanic acid.

22. A method according to claim 21, wherein the nutraceutical contains EGCG in an amount sufficient to provide a daily dosage of 0.3 mg per kg body weight to about 30 mg per kg body weight of the subject to which it is to be administered, pantethine, if present, in an amount sufficient to provide a daily dosage of 1.0 mg per kg body weight to about 50 mg per kg body weight of the subject to which it is to be administered, and phytanic acid, if present, in an amount sufficient to provide a daily dosage of 1.0 mg per kg body weight to about 100 mg per kg body weight of the subject to which it is to be administered.

23. A method according to claim 22, wherein the nutraceutical composition is a food or beverage, or a supplement composition for food or beverage.

24. A method according to claim 22, wherein the nutraceutical composition is a pharmaceutical composition for the treatment of both type 1 and 2 diabetes, and for the prevention of type 2 diabetes in those individuals with pre-diabetes, or impaired glucose tolerance (IGT) or obesity.
